(19) United States
(12) Patent Application Publication
(10) Pub. No.: US 2008/0152669 A1
(43) Pub. Date: Jun. 26, 2008

(54) Method of Stimulating the Production of Mucin in the Eye of a Patient

(76) Inventor: Dharmendra M. Jani, Fairport, NY (US)

Correspondence Address: Bausch & Lomb Incorporated, One Bausch & Lomb Place, Rochester, NY, 14604-2701, US

(21) Appl. No.: 11/943,906

(22) Filed: Nov. 21, 2007

Related U.S. Application Data
(60) Provisional application No. 60/871,020, filed on Dec. 20, 2006.

(51) Int. Cl. A61K 36/02; A61P 27/02

(52) U.S. Cl. 424/195.17

(57) ABSTRACT

The present invention includes a method of treating a patient comprising mucin deficiency comprising administering to an eye of a patient suffering from mucin deficiency, a composition comprising alginate in an amount effective to increase production of mucin in the mucin deficient patient.

### CROSS-REFERENCE

This application claims the benefit of Provisional Patent Application No. 60/871,020 filed Dec. 20, 2006, which is incorporated by reference herein.

### FIELD OF THE INVENTION

This invention relates to a composition for increasing the production of mucin in the eye and a related method of use and method of manufacture. In particular, the invention relates to a method of patients that have a mucin deficiency.

### BACKGROUND

The National Eye Institute/Industry Workshop (1998) defined dry eye as a disease that arises either because of decreased tear production or increased evaporation of tears that results in symptoms of ocular irritation. Recent estimates indicate that 10% to 30% of the adult population suffers from dry eye disease, with the prevalence increasing in older populations. Dry eye is caused by one of three types of deficiencies, mucin deficiency, lipid deficiency and aqueous tear deficiency.

Mucin deficiency occurs due to a failure of goblet cells and/or ocular surface epithelial cells to produce tear mucin. Deficiency of tear mucin destabilizes the tear film. Stevens-Johnson syndrome, burns and pemphigoid are the common causes of mucin deficiency. In the developing world, vitamin A deficiency (xerophthalmia) and trachoma are the most important conditions that affect the mucin layer of the tear film.

Lipid deficiency occurs when the meibomian glands fails to produce anormal amount of lipid. Lipids produced from the meibomian gland contributes to an anterior oily layer of the tear film. The oily layer prevents evaporation of the tear film. The most common causes of tear lipid deficiency include blepharitis and meibomitis. Radiation therapy can cause meibomian gland dropout, leading to a serious deficiency in the tear lipid layer.

Aqueous tear deficiency occurs when the lacrimal gland fails to produce the aqueous portion of the tears. The aqueous layer of the tear film lies in between the lipid and mucin layers and forms the bulk of the tear film. The aqueous layer also dissolves tear mucins, making it more of a gel-like layer.

Dry eye conditions are often treated with a generally aqueous formulation to restore fluid to the eye. A humectant is present in the formulation to assist in the retention of water. Humectants include non-polymeric polyols because of their lubricious nature and ability to retain water. Polymeric humectants such as hydroxypropylmethylcellulose, carboxymethylcellulose, hyaluronic acid, polyacrylic acid and alginate are useful because they increase the viscosity of the formulation. As a result the resident time is improved.

Alginate, for the purpose of this application is a polysaccharide that comprises β-D-mannuronic acid and α-L-guluronic acid monomers or salts or derivatives of such acids or salts.

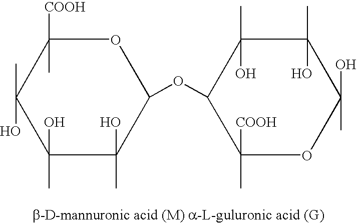

Some alginate polymers are block copolymers with blocks of the guluronic acid (or salt) monomers alternating with blocks of the mannuronic acid (or salt) monomers. See Haug, A. et al., Acta Chem Scand 20:183-190 (1966). Alginate polymers have viscoelastic rheological properties and other properties that make them suitable for some medical applications. See Klock, G. et al., Biocompatibility of manurononic acid-rich alginates, Biomaterials 18(10): 707-713 (1997).

The use of alginate as a thickener for topical ophthalmic use is disclosed in U.S. Pat. No. 6,399,605 and U.S. Publication 2003-0232089 incorporated herein by reference in their entirety. In U.S. Pat. No. 5,776,445, alginate is used as a drug delivery agent that is topically applied to the eye.

U.S. Patent Publication No. 2003/0232089 teaches a dry-eye formulation that contains two polymer ingredients including alginate.

WO2005082333 discloses the use of sodium alginate in a viscoelastic formulation for ophthalmic surgery.

U.S. application Ser. No. 11/475,277 filed Jul. 1, 2005 teaches a dry eye formulation comprising alginate and a polyol.

Mirshafiey, et al., “Sodium alginate as a novel therapeutic option in the experimental colitis,” Scandinavian Journ. of Immun., vol 61, pp. 316-321 (2005) establishes that alginate inhibits cytokine, MMP2 and eicosanoid activity in a rat model suggesting that alginate could be useful to reduce inflammation in the colon of a rat.

Barcello, et al., “Mucin secretion is modulated by luminal factors in the Isolated Vascularly Profuse Rat Colon,” Gut, vol. 46, pp. 218-224 (2000) shows that an increase in mucin secretion in the colon of rats that were fed a solution containing 25 mg of alginate per liter of solution.

In view of the above, it would be desirable to provide an eye-drop solution that will stimulate mucin production in the eye of a patient that has mucin deficiency and that is safe, convenient and economical to use. The present invention addresses these and other needs.

### SUMMARY OF THE INVENTION

The present invention includes a method of treating mucin deficiency in the eye, the method comprises administering to an eye of a patient suffering from mucin deficiency, a composition comprising alginate in an amount sufficient to increase the mucin production in a patient. The alginate stimulates the increase in mucin production.

In one embodiment, the patient, without treatment, produces one natural log order less mucin than does the average population.

In another embodiment, the alginate is in an ophthalmically acceptable vehicle.

In still another embodiment, the composition has a viscosity that is a maximum of about 30000 cps.

In one embodiment, the average molecular weight of alginate is a minimum of about 1 kDa and a maximum of about 5000 kDa.

In yet another embodiment, the concentration of alginate is a minimum of about 0.01 wt. % and a maximum of about 5 wt. % based upon the total weight of the composition.

In still another embodiment, the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

Alternatively or optionally, the pH of the composition is a minimum of about 4 and a maximum of about 8.

Typically, wherein the tonicity of the composition is a minimum of about 200 and a maximum of about 400.

It is preferable that the method of treatment results in no less than a ½ natural log order increase in mucin production.

In another embodiment, the present invention includes a composition for treatment of mucin deficiency comprising an aqueous solution of alginate in an amount effective to increase mucin production.

### DETAILED DESCRIPTION OF THE INVENTION

The present invention includes a method of treating mucin deficiency in the eye, the method comprises administering to an eye of a patient suffering from mucin deficiency, a composition comprising alginate in an amount sufficient to increase the mucin production in a patient. The alginate stimulates the increase in mucin production.

The present invention includes alginate. Alginate is a polysaccharide polymer that has a base unit that is represented by the following formula:

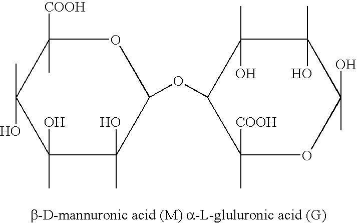

The alginate of one embodiment has a molecular weight that is a minimum of about 1 kDa, about 80 kDa, about 100 kDa, about 500 kDa and/or a maximum of about 5000 kDa, about 2000 kDa, about 1000 kDa, about 700 kDa, about 500 kDa, about 200 kDa, about 100 kDa with ophthalmically pure polyol. In one preferred embodiment, the molecular weight is about 225 kDa.

The alginate of one embodiment, has a ratio of guluronic acid monomer units to mannuronic acid monomer units that is a minimum of about 25:75, about 30:70, about 35:65, or about 40:60. The alginate of an embodiment, has a ratio of guluronic acid monomer units to mannuronic acid monomer units that is a maximum of less than 50:50, about 49:51, about 48:52, about 47:53 or about 46:54. In one embodiment, the ratio of guluronic acid monomer units to mannuronic acid units is about 45:55.

The concentration of alginate is a minimum of about 0.01 wt. % and a maximum of about 2 wt. % based upon the total weight of the solution. Typically, the concentration of alginate is a minimum of about 0.05 wt. %, about 0.1 wt. %, about 0.25%, about 0.5 wt. % or about 1 wt. % based upon the total weight of the solution. Typically, the concentration of alginate is a maximum is about 5 wt. %, about 3 wt. %, about 2 wt. %, about 1.5 wt. % and about 1.2 wt. % based upon the total weight of the solution. Preferably, the concentration of alginate is about 0.5 wt. % based upon the total weight of the solution.

In another embodiment, the alginate containing composition is characterized in that it has a Mark-Houwink number that is a minimum of about 0.6. Typically, the Mark-Houwink number is a minimum of about 0.6 and a maximum of about 1.2. In one embodiment, the Mark-Houwink number is about 1.

According to one embodiment, the ratio of alginate to polyol is a minimum of about 1:20, about 1:4, about 1:3, about 1:2, about 2:3 or about 3:4 and/or a maximum of about 20:1. about 4:1, about 3:1, about 2:1, about 3:2 or about 4:3.

In another embodiment, the alginate is harvested from one or more of the following plant species including lessonia nigrescens, macrocystis pyrifera, laminaria digitata, laminaria japonica and durveillea antartica. Preferably, the source for alignate is from one or more plants including lessonia nigrescens and macrocystis pyrifera. Various purity grades of alginate can be obtained from the same seaweed source and any such purified grade could be used for the purpose of this invention. Preferred grades would be the highly biocompatible Ultrapure sodium alginate grade UP-MVM available from FMC Novamatrix, Norway, which contains insignificant amounts of polyphenols or irritant contaminants and endotoxins that could cause undesirable immunogenic response.

The present composition may also contain a disinfecting amount or a preservative of an antimicrobial agent. Antimicrobial agents are defined as organic chemicals that derive their antimicrobial activity through a chemical or physiochemical interaction with the microbial organisms. These include sorbic acid, quaternary ammonium polymers and low and high molecular weight biguanides. For example, biguanides include the free bases or salts of alexidine, chlorhexidine, hexamethylene biguanides and their polymers, and combinations of the foregoing. The salts of alexidine and chlorhexidine can be either organic or inorganic and are typically gluconates, nitrates, acetates, phosphates, sulfates, halides and the like. A preferred polymeric biguanide is poly(hexamethylene biguanide) commercially available from Zeneca, Wilmington, Del. under the trademark Cosmocil™ CQ. Generally, the hexamethylene biguanide polymers, also referred to as poly(aminopropyl biguanide) (PAPB), have molecular weights of up to about 100 kDa. A particularly preferred preservative is alexidine.

If used in the subject solution, the antimicrobial agent should be used in an amount which will preserve or prevent the growth of the microorganism population in the formulations employed. Preferably, a preservative amount is that which will reduce the bacterial bioburden after 28 days each by 3 logs and prevents the growth of fungal bioburden by ±0.5 log. Typically, such agents are present in a minimum concentration of about 0.0001 wt. %, 0.0003 wt. % or 0.0005 wt. % and a maximum concentration of about 0.0005 wt. % or 0.001 wt. % or about 0.005 wt. % based upon the total weight of the composition.

The aqueous solutions employed in this invention may contain additional ingredients described above, one or more other components that are commonly present in ophthalmic solutions, for example, buffers, stabilizers, tonicity agents and the like, which aid in making ophthalmic compositions more comfortable to the user. The aqueous solutions of the present invention are typically adjusted with tonicity agents to approximate the tonicity of normal lacrimal fluids which is equivalent to a 0.9 wt. % solution of sodium chloride or a 2.8 wt. % of glycerol solution. The solutions are made substantially isotonic with physiological saline used alone or in combination; otherwise, if simply blended with sterile water and made hypotonic or made hypertonic, the lenses will lose their desirable optical parameters. Correspondingly, excess salt or other tonicity agents may result in the formation of a hypertonic solution that will cause stinging and eye irritation. An osmolality is a minimum of about 200 mOsm/kg, about 225 mOsm/kg, about 250 mOsm/kg, about 260 mOsm/kg, about 280 mOsm/kg, about 300 mOsm/kg or about 320 mOsm/kg and/or a maximum of about 400 mOsm/kg, about 380 mOsm/kg, about 360 mOsm/kg, about 340 mOsm/kg or about 320 mOsm/kg. Most preferably, the osmolality is about 240 mOsm/kg to about 320 mOsm/kg.

Preferably, the composition of at least one embodiment of the present invention has a low ionic strength. Typically, the composition contains low concentration of mono or divalent cations typically found in tear fluids. Generally, the composition contains a low concentration of one or more of the following cations: Na+, K+, Ca++, Mg++, and Zn++. In one embodiment, the concentration of the mono or divalent cations that are typically found in tear fluids (i.e. Na+, K+, Ca++, Mg++ and Zn++) has a minimum concentration of about 0.001 wt. %, about 0.005 wt. %, about 0.01 wt. % or about 0.1 wt. % and/or a maximum of about 0.1 wt. %, about 0.01 wt. %, about 0.1 wt. %, about 0.05 wt. % or about 0.01 wt. % based upon the total weight of the composition.

The pH of the present composition should be maintained at a minimum of about 4 about 5, about 5.5, about 6, about 6.5 and/or a maximum of about 7.5, about 7.8, about 8, about 8.5. Suitable buffers may be added, such as borate, citrate, bicarbonate, aminoalcohol buffers, MOPS buffer, bicine, tricine, TRIS, BIS/TRIS and various mixed phosphate buffers (including combinations of Na₂HPO₄, NaH₂PO₄ and KH₂PO₄) and mixtures thereof. Borate buffers are preferred, particularly for enhancing the efficacy of PAPB. Preferred combination buffers include borate/phosphate and borate/citrate combination buffers. Generally, buffers will be used in amounts having a minimum of about 0.05 wt. % or about 0.1 wt. % and/or a maximum of about 1.5 wt. % or about 2.5 wt. %.

In addition to buffering agents, in some instances it may be desirable to include sequestering agents in the present solutions in order to bind metal ions, which might otherwise react with the lens and/or protein deposits and collect on the lens. Ethylene-diaminetetraacetic acid (EDTA) and its salts (disodium) are preferred examples. They are usually added in amounts having a minimum of about 0.01 wt. % and/or a maximum of about 0.2 wt. %.

In one embodiment, there is a method of manufacturing a composition for treatment of mucin deficiency. The method of manufacturing comprises adding to an aqueous solution, ophthalmically pure alginate. As indicated above, the present invention is useful for treating mucin deficiency. For that purpose, compositions for use in the present invention may be sold in a wide range of small-volume containers from 1 ml to 30 ml in size. Such containers can be made from HDPE (high density polyethylene), LDPE (low density polyethylene), polypropylene, poly(ethylene terepthalate) and the like. Flexible bottles having conventional eye-drop dispensing tops are especially suitable for use with the present invention.

The above-described solutions, in accordance with the present invention, may be used by instilling, for example, about one (1) or three (3) drops in the affected eye(s) as needed to increase the level of mucin in the eye.

### EXAMPLE 1

### Formulation

The following ingredients and respective amounts are used to make a base formulation in one preferred embodiment:

| | Minimum | Maximum | Preferred |
|---|---|---|---|
| Formulation A | % w/w | % w/w | % w/w |
| Boric Acid | 0.05 | 1 | 0.5 |
| Sodium Borate | 0.05 | 1 | 0.014 |
| Glycerin | 0.01 | 2 | 0.6 |
| Propylene Glycol | 0.01 | 2 | 0.6 |
| Alginate | 0.1 | 1 | 0.25 |
| HAP (30%) | 0.005 | 0.1 | 0.05 |
| Alexidine 2HCl | 1 ppm | 10 ppm | 4 ppm |
| Purified Water | Q.S. to | Q.S. to | Q.S. to |
| | 100% w/w | 100% w/w | 100% w/w |

| Formulation B | mg/gm | % w/w |
|---|---|---|
| Boric Acid | 5 | 0.5 |
| Sodium Borate | 0.14 | 0.014 |
| Glycerin | 6 | 0.6 |
| Propylene Glycol | 6 | 0.6 |
| Alginate | 5 | 0.5 |
| HAP (30%) | 0.5 | 0.05 |
| Alexidine 2HCl | 4 ppm | 4 ppm |
| Purified Water | Q.S. to 1000 mg | Q.S. to 100% |
| | | w/w |

Formulation Process: A volume of purified water that is equivalent to from about 85% to about 90% of the total batch weight (the temperature of purified water should be below 40° C. before add any raw material) is added into an appropriate stainless steel mixing vessel. Preferably, the temperature of the purified water should be below 40° C. during this step. All the liquid ingredients, HAP, glycerin and propylene glycol, are mixed into the water at the same time. Alginate is selected from the Lessonia nigrescens species. Furthermore, the alginate preferably has an average molecular weight of about 225 kDa. Alginate is dry blended with the powder ingredients, boric acid and sodium borate, and the mix is added slowly with continued agitation and mixed thereafter for at least 45 minutes.

After these ingredients are mixed, Alexidine HCl was added. The batch was allowed to mix for at least 30 minutes. The final mix was sterile filtered using a 0.22 μm sterilizing filter. The preparation is ready for packaging, use and storage. Refrigeration is not needed.

### EXAMPLE 2

### Stimulation of Mucin Production

The formulations of Example 1 were administered to one of a group of two patients that suffer from mucin deficiency. The patients receiving alginate drops belong to the study group. The patients in the control group receive artificial tear solution. Both groups receive treatment four-times a day for four days. Following treatment, tear film samples are collected and tested to quantify mucin content. The patients in the test group are expected to have a higher concentration of mucin than the patients in the control group.

While the invention has been described in conjunction with the detailed description and specific examples, this is illustrative only. Accordingly, many alternatives, modifications and variations will be apparent to those skilled in the art in light of the foregoing description and it is, therefore, intended to embrace all such alternatives, modifications and variations as to fall within the spirit and scope of the appended claims.

What is claimed is:

1. A method of treating mucin deficiency in the eye, the method comprises administering to an eye of a patient suffering from mucin deficiency, a composition comprising alginate in an amount sufficient to increase the mucin production in a patient.

2. The method of claim 1, where in the patient, without treatment, produces one natural log order less mucin than does the average population.

3. The method of claim 1, wherein the alginate is in an ophthalmically acceptable vehicle.

4. The method of claim 1, wherein the composition has a viscosity that is a maximum of about 30000 cps.

5. The method of claim 1, wherein the average molecular weight of alginate is a minimum of about 1 kDa and a maximum of about 5000 kDa.

6. The method of claim 1, wherein the concentration of alginate is a minimum of about 0.01 wt. % and a maximum of about 5 wt. % based upon the total weight of the composition.

7. The method of claim 1, wherein the composition further comprises a buffer selected from the group comprising phosphate buffer, borate buffer, MOPS buffer, citrate buffer, an aminoalcohol buffer and combinations thereof.

8. The method of claim 1, wherein a pH of the composition is a minimum of about 4 and a maximum of about 8.

9. The method of claim 1, wherein the tonicity of the composition is a minimum of about 200 and a maximum of about 400 mOsm/kg.

10. The method of claim 1, wherein the method results in no less than a ½ natural log order increase in mucin production

11. A composition for treatment of mucin deficiency comprising an aqueous solution of alginate in an amount effective to increase mucin production.

12. The composition of claim 11, wherein the alginate is in an ophthalmically acceptable vehicle.

13. The composition of claim 11, wherein the composition has a viscosity that is a maximum of about 30000 cps.

14. The composition of claim 11, wherein the average molecular weight of alginate is a minimum of about 1 kDa and a maximum of about 5000 kDa.

15. The composition of claim 11, wherein the concentration of alginate is a minimum of about 0.01 wt. % and a maximum of about 5 wt. % based upon the total weight of the composition.

16. The composition of claim 11, wherein the composition further comprises a buffer selected from the group comprising phosphate buffer, borate buffer, MOPS buffer, citrate buffer, an aminoalcohol buffer and combinations thereof.

17. The composition of claim 11, wherein a pH of the composition is a minimum of about 4 and a maximum of about 8.

18. The composition of claim 11, wherein the tonicity of the composition is a minimum of about 200 and a maximum of about 400 mOsm/kg.

19. A method for manufacturing a composition for treating mucin deficiency in an eye of a subject, the method comprising combining alginate and a pharmaceutically acceptable carrier, wherein the aginate is present in the composition in an amount effective to increase mucin production in the eye of the subject receiving the composition, to produce the composition.

20. The method of claim 19, further comprising adjusting a tonicity of the composition to a value in the range from about 200 to about 400 mOsm/kg.
